SEQUENCE LISTING (6 sequences)

SEQ ID NO: 1 - protein, 22 aa, synthetic construct
GCCSDPRCNYAHPAICGGAAGG

SEQ ID NO: 2 - protein, 22 aa, synthetic construct
GCCSNPVCHLEHSNLCGGAAGG

SEQ ID NO: 3 - protein, 32 aa, synthetic construct
GPYCQKWMQTCDSERKCCEGMVCRLWCKKKLL

SEQ ID NO: 4 - DNA, 66 bp, synthetic construct
  source 1..66: mol_type = other DNA
ggctgctgcagcgatccgcgctgcaactatgcgcatccggcgatttgcggcggcgcggcgggcggc

SEQ ID NO: 5 - DNA, 66 bp, synthetic construct
  source 1..66: mol_type = other DNA
ggctgctgcagcaacccggtgtgccatctggaacatagcaacctgtgcggcggcgcggcgggcggc

SEQ ID NO: 6 - DNA, 96 bp, synthetic construct
  source 1..96: mol_type = other DNA
ggcccgtattgccagaaatggatgcagacctgcgatagcgaacgcaaatgctgcgaaggcatggtgtgccgcctgtggtgcaaaaaaaaactgctg

(19) United States
(12) Patent Application Publication
(10) Pub. No.: US 2025/0382337 A1
(43) Pub. Date: Dec. 18, 2025

(54) CANNABINOID CONOPEPTIDE GENE THERAPIES FOR PAIN

(71) Applicant: UNIVERSITY OF MIAMI, Miami, FL (US)

(72) Inventors: Jacqueline SAGEN, Miami, FL (US); Stanislava JERGOVA, Miami, FL (US)

(21) Appl. No.: 18/877,410

(22) PCT Filed: Jul. 3, 2023
(86) PCT No.: PCT/US2023/069577

§ 371 Date: Dec. 20, 2024

Related U.S. Application Data
(60) Provisional application No. 63/357,899, filed on Jul. 1, 2022.

(51) Int. Cl. C07K 14/435; A61K 38/00; A61P 25/04

(52) U.S. Cl. CPC C07K 14/43504; A61P 25/04; A61K 38/00

(57) ABSTRACT

Cannabinoids are a promising and potent class of agents in the management of pain, and preclinical studies in rodent models suggest that cannabinoids may be particularly potent in relieving neuropathic pain. Despite the potential benefits and value of cannabinoids, clinical acceptance has been limited due to CNS side effects at systemic analgesic doses and the fear of misuse potential. What is needed are novel cannabinoid-acting compositions and methods for treating pain. The present disclosure relates to compositions targeting cannabinoid receptors and uses thereof for treating, preventing, and/or mitigating pain.

### CROSS REFERENCE TO RELATED APPLICATIONS

This application claims the benefit of U.S. Provisional Patent Application Ser. No. 63/357,899 filed Jul. 1, 2022, the disclosure of which is expressly incorporated herein by reference in its entirety.

### FIELD

The present disclosure relates to engineered conopeptides, engineered polynucleotides, engineered cells, and uses thereof for treating and/or preventing pain.

### BACKGROUND

Cannabinoids are a promising and potent class of agents in the management of pain, and preclinical studies in rodent models suggest that cannabinoids may be particularly potent in relieving neuropathic pain. Despite the potential benefits and value of cannabinoids, clinical acceptance has been limited due to CNS side effects at systemic analgesic doses and the fear of misuse potential. What is needed are novel cannabinoid-acting compositions and methods for treating pain.

### SUMMARY

Marine cone snails produce a wealth of diverse and selective peptides (conopeptides) that are promising therapeutics for pain. Since these are peptidergic, long-term local spinal delivery can also be achievable via gene therapy, thereby avoiding widespread side effects. Disclosed herein are cannabinoid-acting conopeptides produced in the venoms of cone snails that can be developed into therapeutic agents, gene therapies, and cell therapies selective for the cannabinoid receptors, such as CB1 and/or CB2.

Accordingly, in some aspects, disclosed herein is an engineered conopeptide, wherein the engineered conopeptide is a cannabinoid receptor agonist (e.g., a cannabinoid receptor type (CB) 1 agonist and/or a CB2 agonist). In some embodiments, the engineered conopeptide is derived from a Conus species, including, for example, C. textile, C. miles, C. quericinus, C. magus, C. geographus, or C. radiatus.

In some embodiments, the engineered conopeptide comprises an amino acid sequence at least 80% identical to SEQ ID NO: 1, SEQ ID NO: 2, or SEQ ID NO: 3, or a fragment thereof. In some embodiments, the engineered conopeptide comprises the amino acid sequence of SEQ ID NO: 1, SEQ ID NO: 2, or SEQ ID NO: 3.

In some examples, the polypeptide or polynucleotide disclosed herein is a multimer (e.g., a polypeptide comprising multiple conopeptide sequences or a polynucleotide that encodes multiple gene copies of the conopeptides). This can enhance analgesic potency of the engineered conopeptides by releasing multiple copies. Accordingly, in some embodiments, disclosed herein is a polypeptide comprising one or more conopeptide sequences, wherein the conopeptide sequence is at least 80% (for example, at least about 80%, about 85%, about 90%, about 95%, or about 98%) identical to SEQ ID NO: 1, SEQ ID NO: 2, or SEQ ID NO: 3, or a fragment thereof.

In some embodiments, the conopeptide is generated by
a) obtaining a venom sample from the Conus species;
b) separating two or more fractions of conopeptides from the venom sample;
c) performing a fluorescent CB (for example, CB1 or CB2) redistribution assay on each of the separated fractions;
d) selecting the fraction with the highest level of fluorescence; and
e) isolating a conopeptide from the selected fraction of step d) thereby generating the conopeptide.

In some embodiments, the method above further comprises:
f) sequencing the isolated conopeptide; and
g) synthesizing conopeptide.

In some aspects, disclosed herein is an engineered polynucleotide comprising a nucleic acid sequence encoding the engineered conopeptide of any preceding aspect. In some embodiments, the nucleic acid sequence is at least 80% identical to SEQ ID NO: 4, SEQ ID NO: 5, or SEQ ID NO: 6, or a fragment thereof. In some embodiments, the nucleic acid sequence is SEQ ID NO: 4, SEQ ID NO: 5, or SEQ ID NO: 6.

In some embodiments, disclosed herein is a polynucleotide comprising one or more conopeptide-coding sequences, wherein the conopeptide-coding sequence is at least 80% (for example, at least about 80%, about 85%, about 90%, about 95%, or about 98%) identical to SEQ ID NO: 4, SEQ ID NO: 5, or SEQ ID NO: 6, or a fragment thereof.

In some aspects, disclosed herein is a vector comprising one or more of the engineered polynucleotides of any preceding aspect. The vector can be a viral vector (including, for example, an adeno-associated virus (AAV) vector, a lentiviral vector, or a herpes simplex virus (HSV) vector) or a non-viral vector (including, for example, a liposome).

In some aspects, disclosed herein is an engineered cell comprising one or more of the engineered polynucleotides of any preceding aspect. In some embodiments, the engineered cell is a neural stem cell, a neural progenitor cell, or an induced pluripotent stem cell (iPSC). In some embodiments, the neural stem cell or neural progenitor cell is derived from an iPSC (e.g., an iPSC transduced with the engineered polynucleotides or vectors disclosed herein). The engineered nucleotides or vectors can be delivered to the cell through, for example, infection, electroporation, or sonication.

In some aspects, disclosed herein is a method of treating pain in a subject in need, comprising administering to the subject a therapeutically effective amount of the engineered conopeptide, the engineered polynucleotide, the vector, and/or the engineered cell of any preceding aspect. In some embodiments, the engineered conopeptide, the engineered polynucleotide, the vector, or the engineered cell is administered via an intraspinal route, an intrathecal route, or an intraganglionic route. In some embodiments, the engineered polynucleotide can be administered through a viral vector or a non-viral vector.

In some embodiments, the engineered conopeptide, the vector, or the engineered cell decreases levels of proinflammatory cytokines (including, for example, IL-1β and/or TNFα) in the subject.

In some aspects, disclosed herein is a method of treating pain in a subject in need, comprising
a) obtaining a venom sample from the Conus species;
b) separating two or more fractions of conopeptides from the venom sample;
c) performing a fluorescent CB (for example, CB1 or CB2) redistribution assay on each of the separated fractions;
d) selecting the fraction with the highest level of fluorescence;
e) isolating a conopeptide from the selected fraction of step d);
f) administering to the subject in need a therapeutically effective amount of the conopeptide of step e).

In some embodiments, step e) further comprises sequencing the isolated conopeptide; and synthesizing to create the engineered conopeptide.

### DESCRIPTION OF DRAWINGS

The accompanying figures, which are incorporated in and constitute a part of this specification, illustrate aspects described below.

FIG. 1 shows continuous infusion of CB1-acting conopeptide sub-fractions reduces spinal cord injury pain. Based on initial in vitro screening, C. textile venom underwent serial subfractioning and in vitro testing for CB1 internalization and analgesic activity. The promising 3Tex2 fraction was infused via intrathecally implanted pumps in rats with spinal cord injury (SCI) pain starting at 4 weeks after SCI when chronic pain reaches maximum severity (tactile and cold allodynia tests). The pumps are actively infusing for 4 weeks and then deplete. Results showed prolonged analgesic effects of the infused 3Tex2 through the duration of the active pump. These were further subfractioned, peptides isolated, and plasmids designed for gene therapies.

FIG. 2 shows design of CB1 conopeptide analgesic vectors (Vector Builder). Vector Builder software was used to design and produce AAV vectors with CMV promotor and EGFP as a tag. Regulatory sites and sequences were checked by the software for the presence of any Stop codons or incomplete sequences. Once verified, sequences were designed and engineered. The sequences in FIG. 2 include GCCSDPRCNYAHPAICGGAAGG (SEQ ID NO: 1), GCCSNPVCHLEHSNLCGGAAGG (SEQ ID NO: 2), ggctgctgcagcgatccgcgctgcaactatgcgcatccggcgatttgcggcggcgcggcgggcggc (SEQ ID NO: 4), and Ggctgctgcagcaacccggtgtgccatctggaacatagcaacctgtgcggcggcgcggcgggcggc (SEQ ID NO: 5).

FIG. 3 shows in vitro testing of CB1 conopeptide analgesic vectors. The designed plasmids (from FIG. 2) were tested by using lipofection of neural progenitor cells (NPCs), supernatant collected, and tested on HEK cells for CB1 internalization. The supernatant from these was loaded into Alzet pumps at 37° C., collected at 2 weeks and 4 weeks for internalization, to test for engineered CB1 peptides stability and longevity in pumps.

FIG. 4 shows conditioned place preference in SCI animals treated with 3Tex2 fraction or supernatant from cells transduced by L914-195 plasmid (Alzet pumps). The conditioned place preference (CPP) test wasrun with 30 mg/kg gabapentin at 8 weeks of CB1 conopeptide infusions (via Alzet pump infusion) and 2 weeks later (10 weeks), following pump depletion. Animals were trained to receive the analgesic gabapentin on the less preferred side of the cage (bright site). After training, a preference for the gabapentin side indicates the presence of ongoing pain. Gabapentin CPP was reduced in CB1 conopeptide treated animals, indicating successful SCI pain reduction and reduced need for gabapentin treatment. At 10 weeks, after cessation of pumps, all animals developed CPP for GBP. *p<0.05 vs zero hypothesis (development of CPP). #p<0.05 vs saline.

FIG. 5 shows analgesic effects of intraspinally injected AAV CB1-conopeptides on SCI pain response. AAVs were injected at 4 weeks following clip compression SCI (at arrows), when hypersensitivity to mechanical, cold, and heat stimuli were maximal. BL: baseline responses before SCI. *p<0.05 compared with control AAV_GFP (color coded for transgene). #p<0.05 between transgene groups. Results showed significant reduction of neuropathic pain responses with the AAV CB1 conopeptide treatment, particularly robust for AAV_L194-195.

FIGS. 6A-6C show retention of CB1 activity in the rat CSF following intraspinal injection of AAV CB1-conopeptides in rats with SCI pain. FIG. 6A shows CB1 internalization levels by CSF taken from SCI rats treated with control AAV_GFP or AAV CB1-conopeptides intraspinally. *, ***p<0.05, 0.001 between indicated groups. FIG. 6B shows CB1 reduced internalization levels following trypsinization of CSF taken from SCI rats treated with AAV CB1-conopeptides intraspinally, similar to levels in control AAV_GFP, indicating the peptidergic nature of the active CB1 transgenes. FIG. 6C shows examples of CB1 internalization by CSF taken from SCI rats treated with control AAV_GFP or AAV CB1-conopeptides intraspinally with and without trypsin pretreatment.

FIGS. 7A-7C show reduction of inflammation in the spinal cord, CSF and serum of rats with SCI pain following intraspinal injection of AAV CB1-conopeptides. FIG. 7A shows effects of AAV CB1-conopeptide intraspinal injection on lumbar spinal levels of proinflammatory cytokines. *, **p<0.05, 0.01 between indicated groups. FIG. 7B shows effects of AAV CB1-conopeptide intraspinal injection on CSF levels of proinflammatory cytokines. *p<0.05 compared with control AAV_GFP. FIG. 7C shows effects of AAV CB1-conopeptide intraspinal injection on serum levels of proinflammatory (IL1β, TNFα) and anti-inflammatory (IL10) cytokines. *, **p<0.05, 0.01 compared with control AAV_GFP. Serum was collected at the end of the study for these assays. These results indicated reduced inflammation by the AAV CB1 conopeptide treatments.

FIG. 8 shows analgesic effects of AAV CB1-conopeptides on SCI pain responses using the intrathecal route of administration. AAVs were injected at 4 weeks following clip compression SCI (at arrows), when hypersensitivity to tactile, cold, and heat stimuli were maximal. BL: baseline responses before SCI. *, **p<0.05, 0.01 compared with control AAV_GFP (color coded for transgene). #p<0.05 between transgene groups. Results showed significant reduction of neuropathic pain responses with the AAV_CB1 conopeptide treatment, particularly robust for AAV_L194-195.

FIGS. 9A-9B show reduction of inflammation in the CSF and serum of rats with SCI pain following intrathecal injection of AAV CB1-conopeptides. FIG. 9A shows effects of AAV CB1-conopeptide intrathecal injection on CSF levels of proinflammatory cytokines. *p<0.05 compared with control AAV_GFP. FIG. 9B shows effects of AAV CB1-conopeptide intrathecal injection on serum levels of proinflammatory cytokines IL1β and TNFα and anti-inflammatory IL10. *p<0.05 **p<0.01 compared with control AAV_GFP.

FIG. 10 shows analgesic effects of intra-ganglionic (DRG) injected AAV CB1-conopeptides on SCI pain. AAVs were injected at 4 weeks following clip compression SCI (at arrows), BL: baseline responses before SCI. Reduced pain responses by the CB1 transgene treatments appear to emerge by 1 week following injection and sustain at least up to 10 weeks post injury. *p<0.05, **p<0.01 vs GFP.

FIGS. 11A-11B show reduction of inflammation in the CSF and serum of rats with SCI pain following DRG injection of AAV CB1-conopeptides. FIG. 11A shows effects of AAV CB1-conopeptide after DRG injection on CSF levels of proinflammatory cytokines. *p<0.05, **p<0.01 compared with control AAV_GFP. FIG. 11B shows effects of AAV CB1-conopeptide intrathecal injection on serum levels of proinflammatory cytokines IL1β and TNFα and anti-inflammatory IL10. *p<0.05 **p<0.01 compared with control AAV_GFP.

FIGS. 12A-12C mechanistic evaluation: reversal of AAV CB1-conopeptide analgesic effects with CB1 antagonist. The effect of injection (i.p) of CB1 receptor antagonist AM251 (3 mg/kg, ip) on tactile and cold hypersensitivity of SCI animals following treatment with spinal (FIG. 12A), DRG (FIG. 12B), or intrathecal injection (FIG. 12C) of AAV encoding L914-195 and L914-185. Sensitivity was analyzed 15 min post injection. *, **p<0.05, 0.01 between indicated groups. Results supported that AAV CB1 conopeptide transgene analgesic effects were mediated via CB1 receptors.

FIGS. 13A-13B show Conditioned Place Preference (CPP) in SCI animals treated with AAV CB1 conopeptides (DRG injection). (FIG. 13A) Animals trained with saline do not show any place preference. Treatment with gabapentin (GBP, 30 mg/kg, i.v., 3 days) induced place preference in GFP animals (#p<0.05). Reduced preference for this drug paired side was observed for animals treated with L914-195 construct, tested 7 weeks post SCI. (FIG. 13B) Similar experiment with animals pretreated with AM 251 before each GBP injection. Pretreatment led to development of place preference in groups treated with L914-195 construct, indicating that the effect of the active CB1 substance generated by the construct is antagonized by AM 251, CB1 antagonist, tested 8.5 weeks post SCI.

FIGS. 14A-14B show reduced glial activity in spinal cord of SCI animals by AAV CB1 conopeptide treatment. Immunohistochemical staining shows reduced density of glial cells, both astrocytes (GFAP) and microglia (Iba-1), in animals treated by L914 constructs compared to GFP animals, showing reduced SCI spinal scarring and inflammation. Analysis of GFAP (left) and Iba-1 (right) density in stained spinal slides, *p<0.05 vs GFP, #p<0.05, ##p<0.01 vs sham.

FIG. 15 shows detection of L914-195_GFP tagged construct in the spinal cord of SCI animals after various routes of injection. Immunohistochemistry for neuronal marker NeuN and GFP fluorescence were used to detect L914-195_GFP labelled construct in the spinal tissue and to evaluate its distribution.

FIG. 16 shows RNA-in situ hybridization to detect L914-195 construct in the spinal cord. RNA-in situ hybridization was conducted to further confirm the presence of the L914-195 construct in the absence of custom-made antibody. A small sample of RNA probes was designed by Vector Builder. Probes were labeled by using FISH Tag RNA kit (ThermoFisher), to incorporate amine-modified nucleotide into RNA, followed by fluorescent labeling with an amine-reactive Alexa Fluor dye and purification of the labeled probe using PureLink nucleic acid purification technology. DAPI shows cell nuclei.

FIG. 17 shows detection of RNA_L914-195 in the spinal cord of SCI animals after various routes of injection. RNA-in situ hybridization showing the dorsal horn distribution of the presence of the L914-195 RNA following the intra-spinal, intrathecal, and intra-DRG routes of injections of the AAV_L914-195 (CB1 conopeptide).

FIGS. 18A-18C show distribution analysis of in-situ RNA signal and GFP tagged L914-195 construct in the lumbar spinal cord. Rostral-caudal distribution of L914-195 RNA following the intra-spinal, intrathecal, and intra-DRG routes of injections of the AAV_L914-195 (CB1 conopeptide). This is shown in conjunction with distribution of GFP_L914-195 peptide. Results show circumscribed transgene expression with little diffusion, supporting the ability to selectively target spinal pain processing centers with minimal risk to off-target exposure.

FIGS. 19A-19B show pain reduction by AAV_CB1 conopeptide in other chronic pain conditions. In the chronic constriction injury (CCI) model (a model for peripheral neuropathic pain), tactile hypersensitivity and cold hypersensitivity were reduced by the CB1 conopeptides, particularly the L914-195. Studies in the monoiodoacetate model (knee injection to induce osteoarthritis-like knee pain), both CB1 conopeptides also attenuated pain symptoms. In both models, intrathecal injections of the AAV_CB1 conopeptides were given 1 week post-injury (at arrows). These findings show that the CB1 conopeptide gene therapy can reduce chronic pain of several distinct etiologies.

FIG. 20 shows design of CB2 conopeptide analgesic vectors. The sequences in FIG. 20 include GPYCQKWMQTCDSERKCCEGMVCRLWCKKKLL (SEQ ID NO: 3), Ggcccgtattgccagaaatggatgcagacctgcgatagcgaacgcaaatgctgcgaaggcatggtgtgccgcctgtggtgcaaaaaaaa actgctg (SEQ ID NO: 6).

FIG. 21 shows effect of intraspinally injected AAV CB2-conopeptide on SCI pain. Left panels: Effects of AAV CB2-conopeptide CGF2 intraspinal injection on SCI neuropathic pain responses. AAVs were injected at 4 weeks following clip compression SCI (at arrows), when hypersensitivity to cold and heat stimuli were maximal. BL: baseline responses before SCI. *p<0.05 compared with control AAV_GFP. Upper right: CB2 internalization levels by CSF taken from SCI rats treated with control AAV_GFP or AAV CB2-conopeptides intraspinally at 1 or 4 weeks following injection. **p<0.01 between indicated groups. Lower right panel: CB2 internalization levels following trypsinization of CSF from the same groups. Results show transient stability in contrast to the AAV CB1 conopeptides.

FIGS. 22A-22C show identification of other CB2 agonists within venom fractions C. Rad3 and C. Mil6. (FIG. 22A) Internalization of CB2 receptor was induced after incubation of cells with C.Rad3 or C. Mil6 fractions and reduced after pretreatment with CB2 antagonists AM630. (FIG. 22B) Internalization with precipitated fractions of C. Rad3 and C. Mil6 was abolished by trypsin pretreatment, indicating the presence of peptides within fractions with CB2 activity. (FIG. 22C) Right Top: Immunohistochemistry of HEK CB2 cells after treatment with C. Rad3 (left) or C. Mil6 (right) immunoprecipitants. Right Bottom: Trypsin pretreatment reduced internalization of CB2 receptor *p<0.05, **p<0.01, ***p<0.001 between indicated groups.

FIG. 23 shows analgesic effect of immunoprecipitated fractions of C. Rad3 and C. Mil6 after intrathecal injection in SCI animals. To evaluate initial potency of the CB2 agonist conopeptides of C.Rad3 and C. Mil6, 5 uL (2 ug/ml) were injected via intrathecal catheter for 3 days (starting 4 weeks post-SCI) and tested for changes in pain responses post-injection on days 1-3 and 7. The first test was conducted at the same day of the last injection (Day 3 of the injection pattern=Day 0 for the post-injection testing schedule). Findings showed good anti-allodynic activity to tactile and cold stimuli for both C Rad3 and C Mil6 compared with saline, and retained reduced pain responses for up to 3 days following the last injection. *p<0.05, **p<0.01 vs saline control.

FIG. 24 shows reduced glial activity in spinal cord of SCI animals by CB2 conopeptide treatment. Both C.Rad3 and C. Mil6 reduced spinal cord astrogliosis in parallel with their pain-reducing activity in SCI animals (*p<0.05, **p<0.01 vs saline control).

FIG. 25 shows analgesic effect of combined C. Rad3 and C. Mil6 after intrathecal injection in SCI animals. Studies are being conducted to determine whether enhanced analgesic activity can be obtained using combinations of the most promising CB conopeptides. As an example of this, C.Rad3 and C. Mil6 were administered in combination and delivered intrathecally by continuous infusion for 1 week (starting 4 weeks post-SCI) using an implanted Alzet pump. Findings showed robust anti-allodynic activity to tactile and cold stimuli with this co-administration. Thus, combination constructs encoding both CB2 conopeptides (as well as CB1-CB2 conopeptide combination constructs) are being synthesized. *p<0.05, **p<0.01, ***p<0.001 vs saline control.

FIG. 26 shows engineering stem cells with AAV CB conopeptide vectors for delivery of analgesic conopeptides via cell transplantation. Ratneural progenitor cells, NPCs and human induced pluripotent cells (hiPSCs) differentiated to GABA NPCs were used.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation, intraspinal, intrathecal, intraganglionic and the like. Administration includes self-administration and the administration by another.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., chronic pain). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises the polypeptides, the polynucleotides, the engineered cells disclosed herein.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “control” is an alternative subject or sample used in an experiment for comparison purposes. A control can be “positive” or “negative.”

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant polypeptide” refers to an amount of a recombinant peptide sufficient to prevent, treat, or mitigate pain.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

The term as used herein “engineered” and other grammatical forms thereof may refer to one or more changes of nucleic acids or amino acids. The term “engineered” may refer to a change, addition and/or deletion of one or more nucleotides or one or more amino acid residues. Engineered cells can also refer to cells that contain added, deleted, and/or changed genes.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, “induced pluripotent stem cells” or “iPSC” are cells that are differentiated, somatic cells reprogrammed to pluripotency. The cells are substantially genetically identical to their respective differentiated somatic cells of origin and display characteristics similar to higher potency cells, such as ES cells. See, Yu J, et al., “Induced pluripotent stem cell lines derived from human somatic cells,” Science 318:1917-1920 (2007), incorporated herein by reference as if set forth in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

The term “nucleic acid” as used herein means a polymer composed of nucleotides, e.g., deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g., promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Therapeutically effective amount” refers to the amount of a composition that will elicit the biological or medical response of a tissue, system, animal, or human that is being sought by the researcher, veterinarian, medical doctor or other clinician over a generalized period of time. In some embodiments, a desired response is reduction of pain in a subject. In some embodiments, the desired response is prevention, treatment, and/or mitigation of pain or the related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount as described herein can be determined by one of ordinary skill in the art.

A therapeutically significant reduction in a symptom is, e.g., at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. It will be understood that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of an inflammatory disease or condition and/or alleviating, mitigating or impeding one or more causes of an inflammatory disease. Treatments according to the invention may be applied preventively, prophylactically, palliatively or remedially. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

Disclosed herein are the components to be used to prepare the disclosed compositions as to be used in the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### Compositions

In some aspects, disclosed herein is an engineered conopeptide, wherein the engineered conopeptide is a cannabinoid receptor agonist (e.g., a cannabinoid receptor type (CB) 1 agonist and/or a CB2 agonist). In some embodiments, the engineered conopeptide is derived from a Conus species, including, for example, C. textile, C. miles, C. quericinus, C. magus, C. geographus, or C. radiatus.

“CB1” refers herein to a polypeptide that, in humans, is encoded by the CNR1 gene. In some embodiments, the CB1 polypeptide is that identified in one or more publicly available databases as follows: HGNC: 2159, NCBI Gene: 1268, Ensembl: ENSG00000118432, OMIM®: 114610, UniProtKB/Swiss-Prot: P21554.

“CB2” refers herein to a polypeptide that, in humans, is encoded by the CNR2 gene. In some embodiments, the CB2 polypeptide is that identified in one or more publicly available databases as follows: HGNC: 2160, NCBI Gene: 1269, Ensembl: ENSG00000188822, OMIM®: 605051, UniProtKB/Swiss-Prot: P34972.

In some embodiments, the engineered conopeptide comprises an amino acid sequence at least 80% (for example, at least about 80%, about 85%, about 90%, about 95%, or about 98%) identical to SEQ ID NO: 1, SEQ ID NO: 2, or SEQ ID NO: 3, or a fragment thereof. In some embodiments, the engineered conopeptide comprises the amino acid sequence of SEQ ID NO: 1, SEQ ID NO: 2, or SEQ ID NO: 3.

In some embodiments, the conopeptide is generated by
a) obtaining a venom sample from the Conus species;
b) separating two or more fractions of conopeptides from the venom sample;
c) performing a fluorescent CB (for example, CB1 or CB2) redistribution assay on each of the separated fractions;
d) selecting the fraction with the highest level of fluorescence; and
e) isolating a conopeptide from the selected fraction of step d) thereby generating the conopeptide.

In some embodiments, the method above further comprises:
f) sequencing the isolated conopeptide; and
g) synthesizing conopeptide.

Separation of fractions from the venom sample in Step b) can comprise using HPLC, MS, or other methods. The conopeptides isolated from the selected fractions are surprisingly effective in controlling pain.

In some aspects, disclosed herein is an engineered polynucleotide comprising a nucleic acid sequence encoding the engineered conopeptide of any preceding aspect. In some embodiments, the nucleic acid sequence is at least 80% (for example, at least about 80%, about 85%, about 90%, about 95%, or about 98%) identical to SEQ ID NO: 4, SEQ ID NO: 5, or SEQ ID NO: 6, or a fragment thereof. In some embodiments, the nucleic acid sequence is SEQ ID NO: 4, SEQ ID NO: 5, or SEQ ID NO: 6.

It is herein contemplated that the conopeptides and polynucleotides disclosed can be modified to prolong their activity, stabilize and reduce degradation, and increase analgesic potency, etc. In some examples, the polypeptide or polynucleotide disclosed herein is a multimer (e.g., a polypeptide comprising multiple conopeptide sequences or a polynucleotide that encodes multiple gene copies of the conopeptides). This can enhance analgesic potency of the engineered conopeptides by releasing multiple copies. Accordingly, in some embodiments, disclosed herein is a polypeptide comprising one or more conopeptide sequences, wherein the conopeptide sequence is at least 80% (for example, at least about 80%, about 85%, about 90%, about 95%, or about 98%) identical to SEQ ID NO: 1, SEQ ID NO: 2, or SEQ ID NO: 3, or a fragment thereof. In some embodiments, disclosed herein is a polynucleotide comprising one or more conopeptide-coding sequences, wherein the conopeptide-coding sequence is at least 80% (for example, at least about 80%, about 85%, about 90%, about 95%, or about 98%) identical to SEQ ID NO: 4, SEQ ID NO: 5, or SEQ ID NO: 6, or a fragment thereof.

In some aspects, disclosed herein is a vector comprising one or more (e.g., one or more, two or more, or three or more) of the engineered polynucleotides of any preceding aspect. The vector can be a viral vector (including, for example, an adeno-associated virus (AAV) vector, a lentiviral vector, or a herpes simplex virus (HSV) vector) or a non-viral vector (including, for example, a liposome).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. “Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transporting into a nucleus of a target cell. The term “viral vector” is also meant to refer to those forms described more fully in U.S. Patent Application Publication U.S. 2018/0057839, which is incorporated herein by reference in its entirety. Suitable viral vectors include, e.g., adenoviruses, adeno-associated virus (AAV), vaccinia viruses, herpesviruses, baculoviruses, retroviruses, parvoviruses, herpes simplex virus vectors, lentiviruses, and he like. Methods for gene delivery are known in the art. See, e.g., U.S. Pat. Nos. 5,399,346, 5,580,859, 5,589,466, incorporated by reference herein in their entireties.

In some aspects, disclosed herein is an engineered cell comprising one or more of the engineered polynucleotides of any preceding aspect. In some embodiments, the engineered cell is a neural stem cell, a neural progenitor cell, or an induced pluripotent stem cell (iPSC). In some embodiments, the neural stem cell or neural progenitor cell is derived from an iPSC (e.g., an iPSC transduced with the engineered polynucleotides or vectors disclosed herein).

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

In certain embodiments, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR. In some embodiments, regulatable vectors are used herein for the transgenes to be turned on and off (including, for example, Tet-on/off systems or inflammation inducible systems such as NfKB promoter).

In certain embodiments, the disclosure contemplates pharmaceutical compositions comprising the conopeptides, the polynucleotides, the vectors, or engineered cells disclosed herein, or optionally other pharmaceutical agent, or pharmaceutically acceptable salts thereof, and a pharmaceutically acceptable excipient. In certain embodiments, this disclosure contemplates the production of a medicament comprising polypeptides, polynucleotides, vectors, or engineered cells disclosed herein, or agents disclosed herein and uses for methods disclosed herein.

The compositions disclosed herein may be in solution, suspension (for example, incorporated into microparticles (such as exosomes) or liposomes). These may be targeted to a particular cell type via antibodies, receptors, or receptor ligands. The compositions disclosed herein may be in exosomes. The term “exosome”, as used herein, refers to a cell-derived membranous vesicle. They refer to extracellular vesicles, which are generally of between 30 and 200 nm in size, for example in the range of 50-100 nm in size. The exosomes can be engineered to express one or more ligands or molecules for cell-targeting (e.g., CNS-targeting) delivery.

Drug load or loading capacity refers to the amount of the composition that can be present in the exosome can be from about 0.1% to about 60% of its exosome weight. For example, the amount of the composition present in the exosome can be from about 0.1%, about 0.2%, about 0.3%, about 0.4%, about 0.5%, about 0.6%, about 0.7%, about 0.8%, about 0.9%, about 1%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 15%, about 16%, about 17%, about 18%, about 19%, about 20%, about 22%, about 24%, about 26%, about 28%, about 30%, about 32%, about 34%, about 36%, about 38%, about 40%, about 55%, or about 60% of its exosome weight.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### Methods of Treatment

In some aspects, disclosed herein is a method of treating pain (e.g., chronic pain) in a subject in need, comprising administering to the subject a therapeutically effective amount of the engineered conopeptide, the engineered polynucleotide, the vector, and/or the engineered cell disclosed herein.

In some embodiments, the engineered conopeptide comprises an amino acid sequence at least 80% (for example, at least about 80%, about 85%, about 90%, about 95%, or about 98%) identical to SEQ ID NO: 1, SEQ ID NO: 2, or SEQ ID NO: 3, or a fragment thereof. In some embodiments, the engineered conopeptide comprises the amino acid sequence of SEQ ID NO: 1, SEQ ID NO: 2, or SEQ ID NO: 3.

In some embodiments, the engineered polynucleotide comprises a nucleic acid sequence encoding the engineered conopeptide of any preceding aspect. In some embodiments, the nucleic acid sequence is at least 80% (for example, at least about 80%, about 85%, about 90%, about 95%, or about 98%) identical to SEQ ID NO: 4, SEQ ID NO: 5, or SEQ ID NO: 6, or a fragment thereof. In some embodiments, the nucleic acid sequence is SEQ ID NO: 4, SEQ ID NO: 5, or SEQ ID NO: 6.

In some embodiments, the vector comprises one or more (e.g., one or more, two or more, or three or more) of the engineered polynucleotides of any preceding aspect. The vector can be a viral vector (including, for example, an adeno-associated virus (AAV) vector, a lentiviral vector, or a herpes simplex virus (HSV) vector) or a non-viral vector (including, for example, a liposome).

In some embodiments, the engineered cell comprises one or more of the engineered polynucleotides or vectors disclosed herein. In some embodiments, the engineered cell is a neural stem cell, a neural progenitor cell, or an induced pluripotent stem cell (iPSC). In some embodiments, the neural stem cell or neural progenitor cell is derived from an iPSC (e.g., an iPSC transduced with the engineered polynucleotides or vectors disclosed herein). The engineered nucleotides or vectors can be delivered to the cell through infection, electroporation, or sonication.

In some embodiments, the engineered conopeptide, the vector, or the engineered cell decreases levels of proinflammatory cytokines (including, for example, IL-1β and/or TNFα) in the subject.

Also disclosed herein is a method of treating pain in a subject in need, comprising
a) obtaining a venom sample from the Conus species;
b) separating two or more fractions of conopeptides from the venom sample;
c) performing a fluorescent CB (for example, CB1 or CB2) redistribution assay on each of the separated fractions;
d) selecting the fraction with the highest level of fluorescence;
e) isolating a conopeptide from the selected fraction of step d);
f) administering a therapeutically effective amount of the conopeptide of step e) into the subject in need.

The pharmaceutical compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. In some embodiments, the engineered conopeptide, the engineered polynucleotide, the vector, or the engineered cell is administered via an intraspinal route, an intrathecal route, or an intraganglionic route to limit distribution of the administered compositions, thereby minimizing any adverse side effects. In some embodiments, the engineered polynucleotide can be administered through a viral vector or a non-viral vector. In some embodiments, the methods of treatment disclosed herein comprise administering to the subject (e.g., via a systemic administration) the vectors or polynucleotides disclosed herein and then applying focused ultrasound to directly target vectors or polynucleotides to specific brain and/or spinal regions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

In some embodiments, dosing frequency for the composition disclosed herein, includes, but is not limited to, no more than once every 30 years, every 25 years, every 20 years, every 15 years, every 10 years, every 5 years, every 4 years, every 3 years, every 2 years, every 12 months, or every 6 months.

Dosing frequency for the composition disclosed herein, includes, but is not limited to, at least once every 30 years, every 25 years, every 20 years, every 15 years, every 10 years, every 5 years, every 4 years, every 3 years, every 2 years, every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for the composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is constant. For example, the administration can be carried out daily, every two days, every three days, every four days, every five days, or weekly. Administration can also be continuous and adjusted to maintaining a level of the compound within any desired and specified range. It should be understood and herein contemplated that the compositions disclosed herein can be used in combination with a pain reliever and, in some examples, reduce the dosing frequency of the pain reliever.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

The venom extracts of six Conus species (C. textile (tex), C. miles (mil), C. quericinus (que), C. magus (mag), C. geographus (geo), and C. radiatus (rad)) were provided for initial screening. CB1 and CB2 cells were routinely grown to screen for CB receptor internalization using standard synthetic cannabinoid agonists WIN 55,212-2 and CP55,940. Selectivity for CB1 or CB2 receptors was tested by adding CB1-selective antagonist AM251 or CB2-selective antagonist AM630. Promising subfractions of CB-active conopeptide fractions were selected based on in vitro and in vivo assays. This study identified and selected subfractions of C. Tex and C. Geo, and had them further fractionalized by HPLC analysis, with resulting subfractions then again tested in vitro for the CB1/2 receptor internalization properties. The CB1 and CB2 receptor agonists were isolated from the subfractions by immunoprecipitation using Sepharose beads. With the last fraction series of C. Tex and C. Geo, an optimized immunoprecipitation protocol was used for isolation and purification of the CB1 and CB2 active components from Conus venoms. After the elution, the samples were purified with gel electrophoresis and the respective bands were identified with Coomasie staining. To prepare samples for mass spec analysis, the protocol was followed for in gel digestion provided to us by the MS core. Samples were analyzed by MS analysis.

The resultant promising CB1 and CB2 subfractions were tested in a rat model of neuropathic pain following spinal cord injury, by intrathecal infusion with Alzet minipumps. Reduced neuropathic pain symptoms observed during the intrathecal infusion period (up to 4 weeks with the Alzet pump model 2004 (volume 200 uL, rate 0.25 uL/h). More robust analgesic effects were observed with the C. Tex infusion (CB1-selective) and this was prolonged for several weeks following pump depletion.

Thus, two samples of C. Tex subfractions (L914-185 and L914-195) from above yielding promising results, with 2 sequences identified by Phyre2 (Imperial College London), Swiss Model (Univ. of Basel) and Sequence Manipulation Suite (Stothard P (2000; The Sequence Manipulation Suite: JavaScript programs for analyzing and formatting protein and DNA sequences. Biotechniques 28:1102-1104) software).

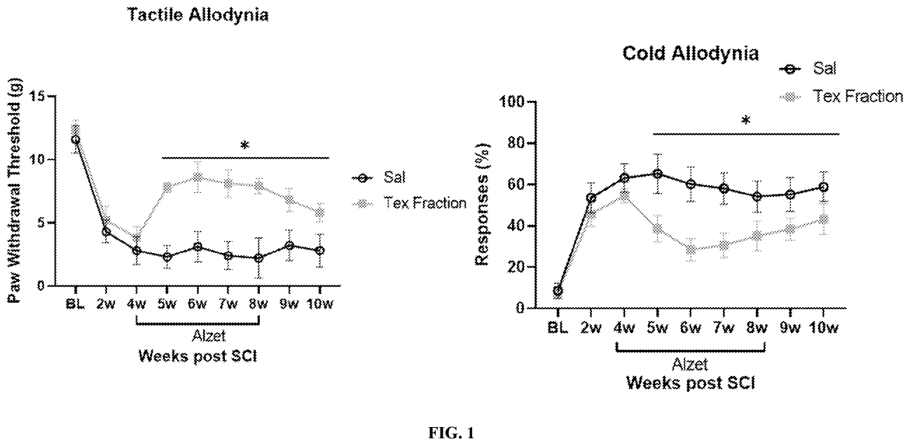

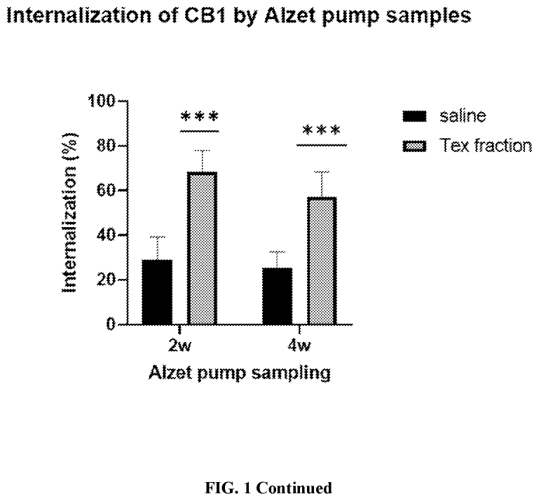

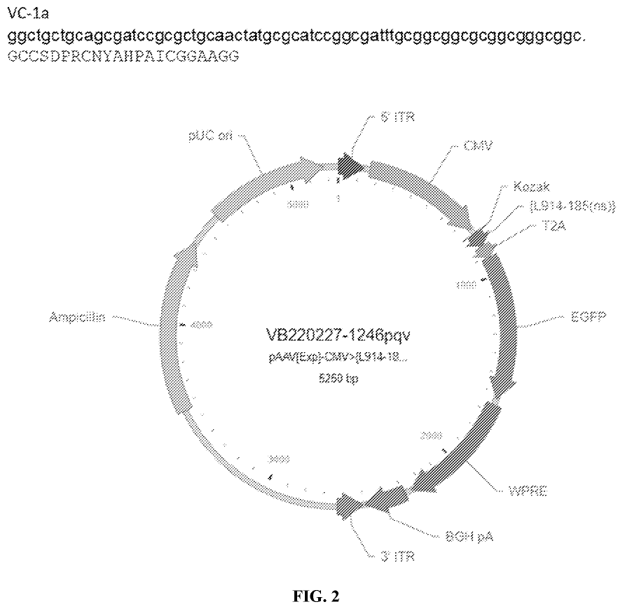

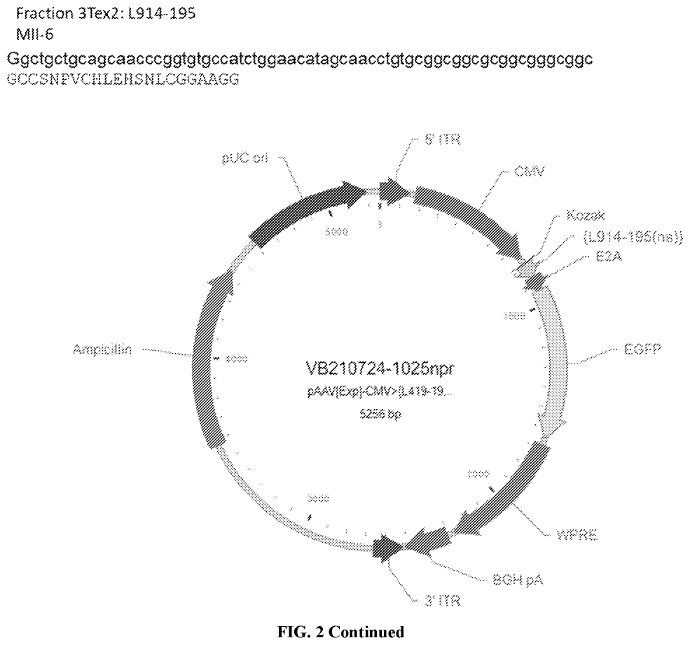

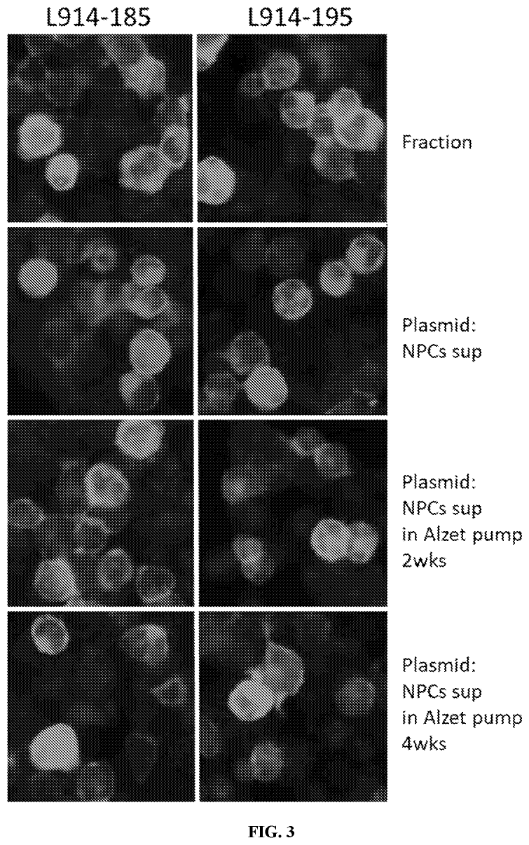

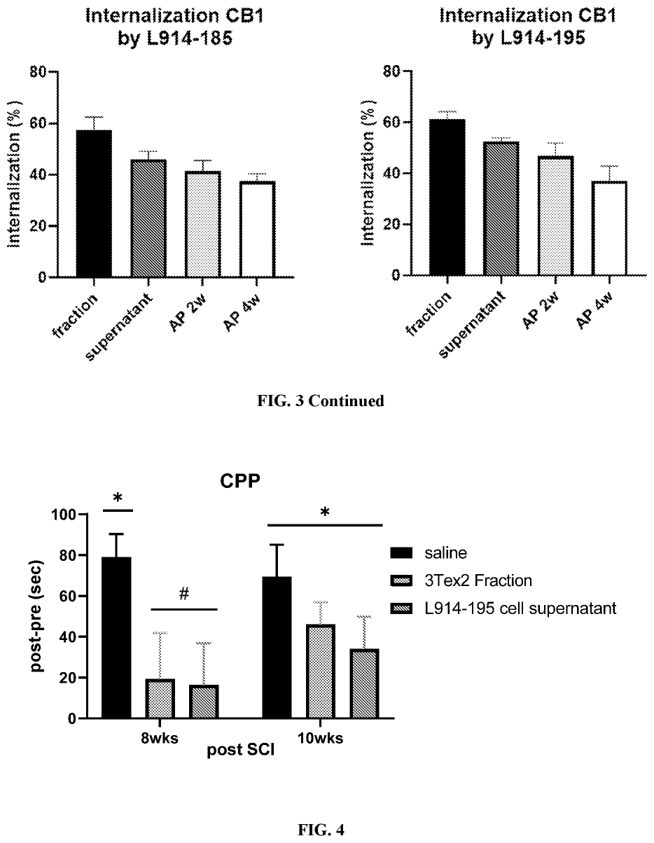

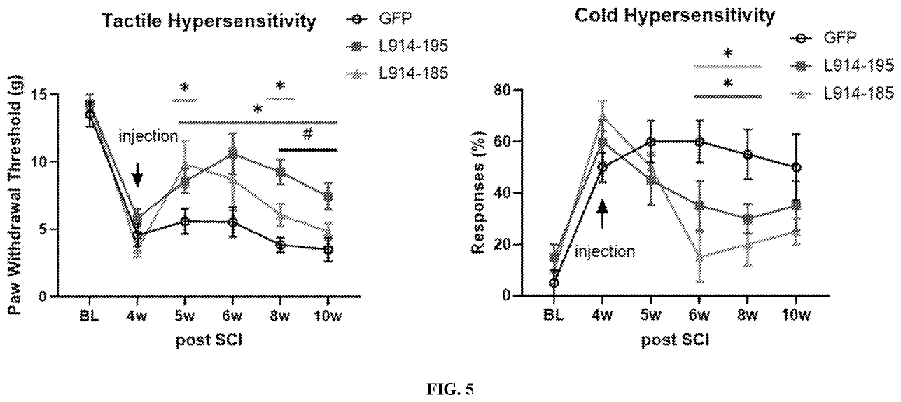

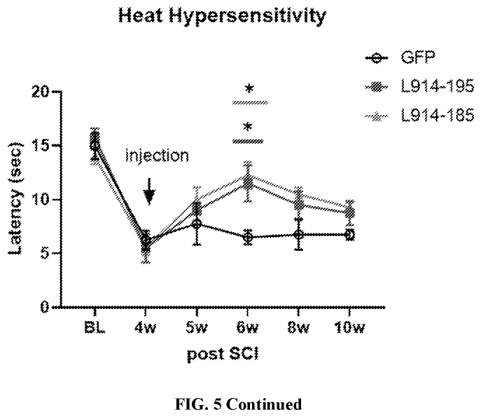

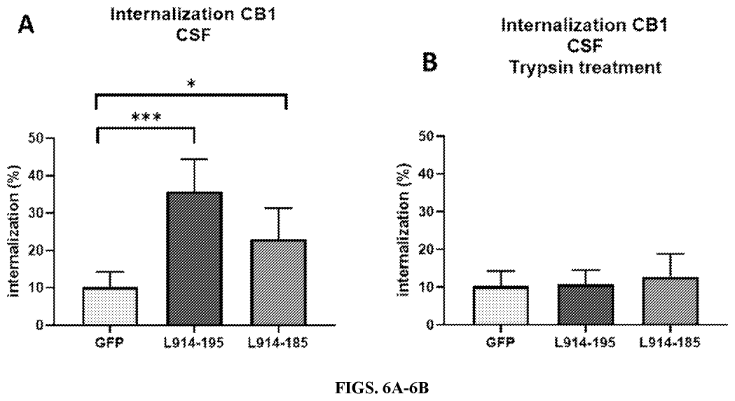

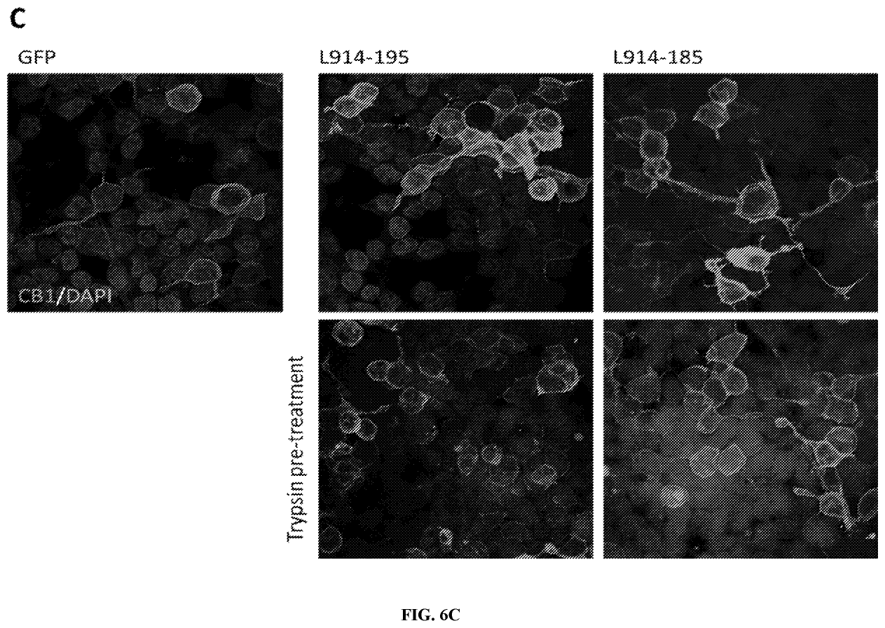

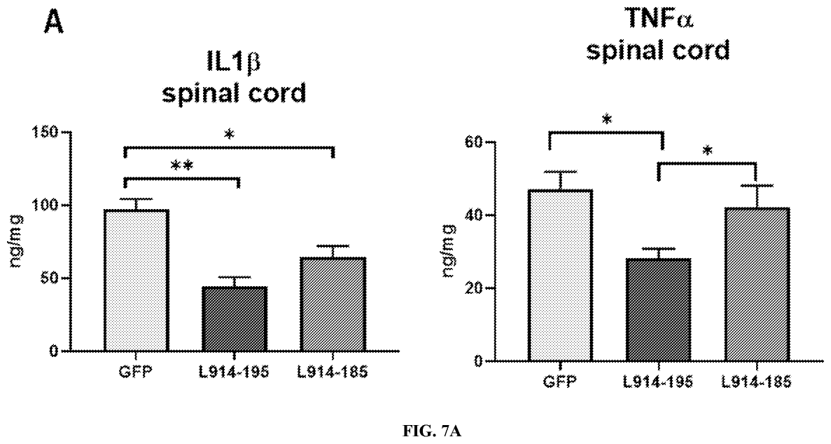

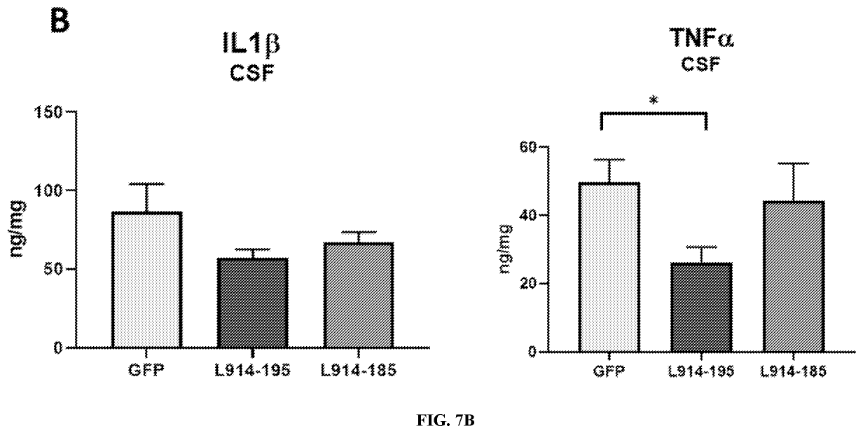

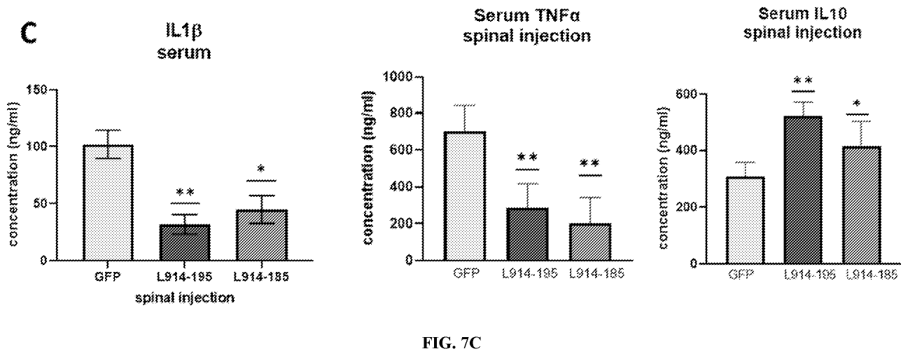

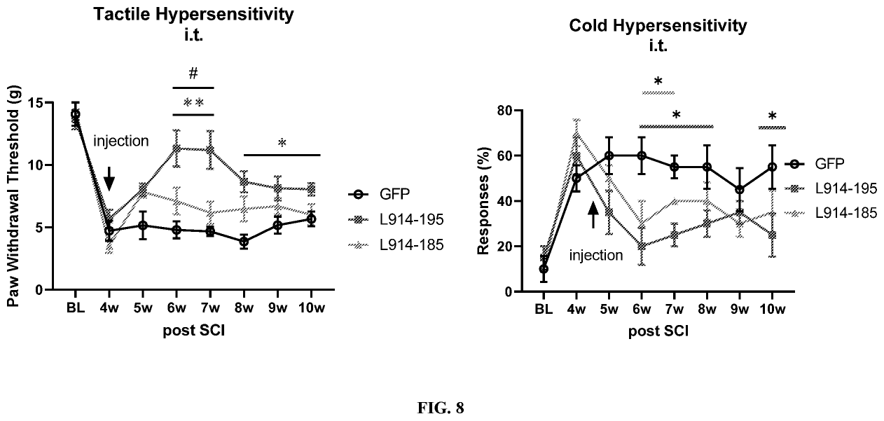

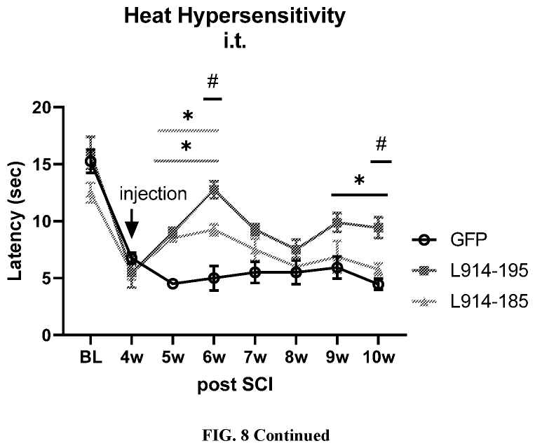

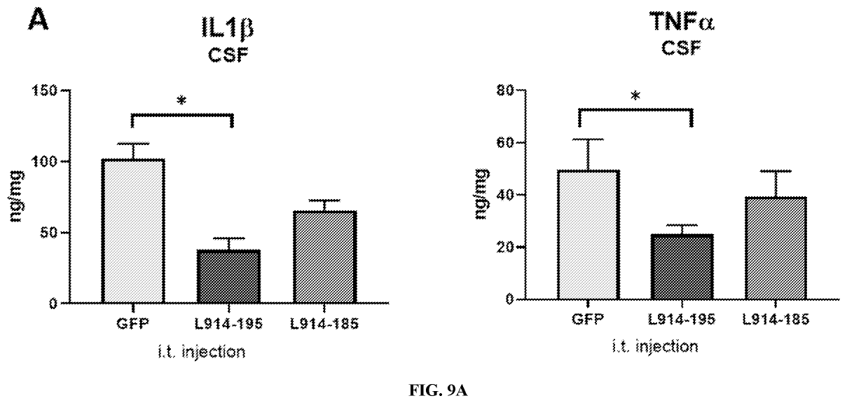

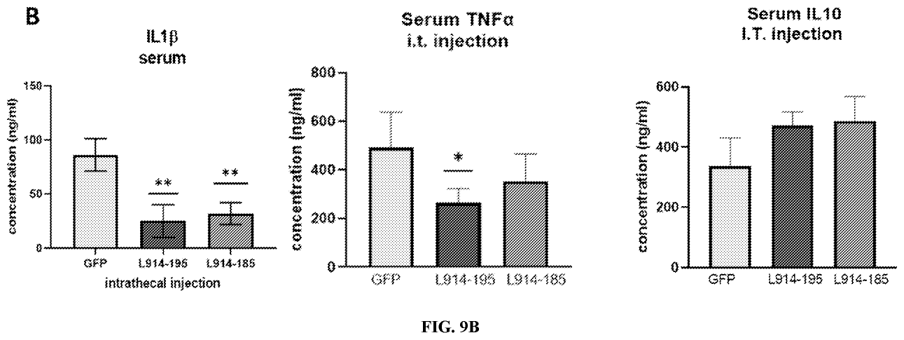

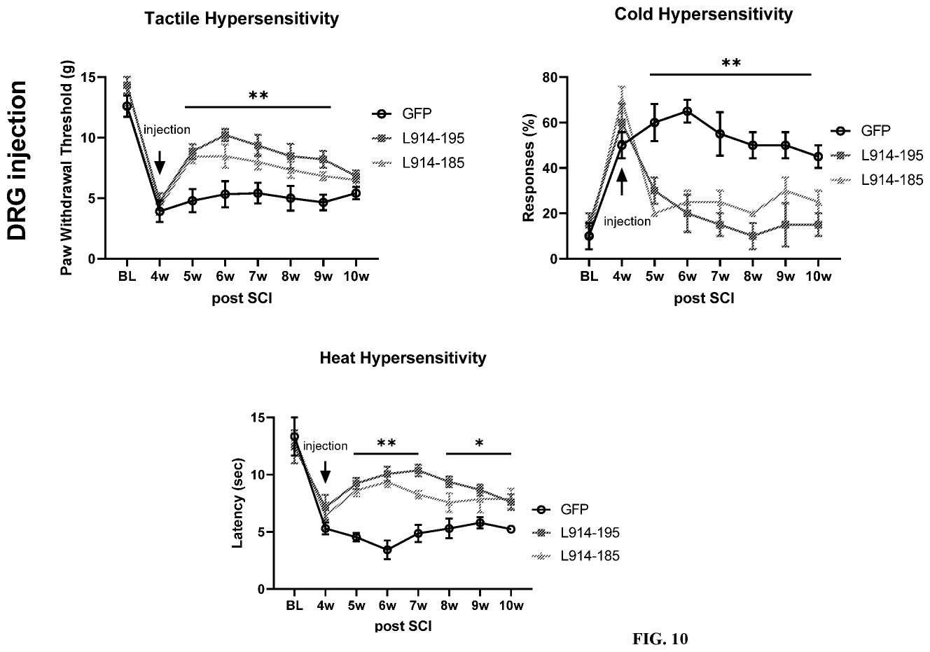

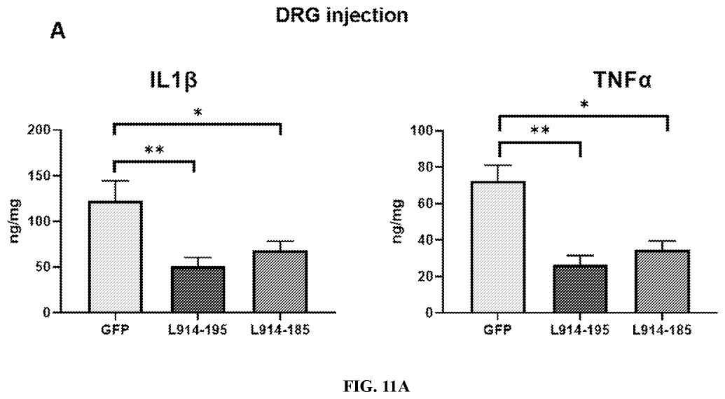

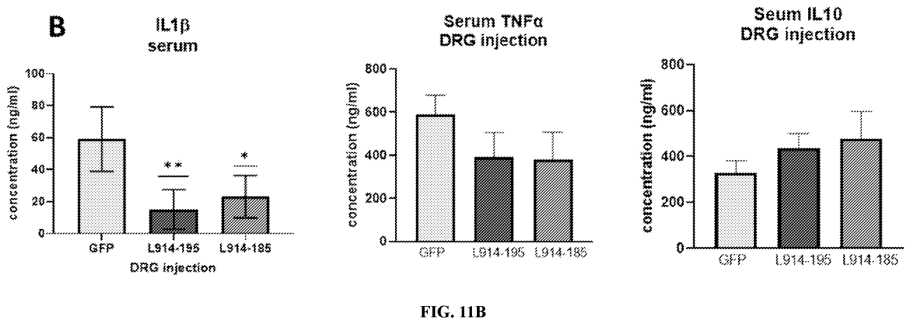

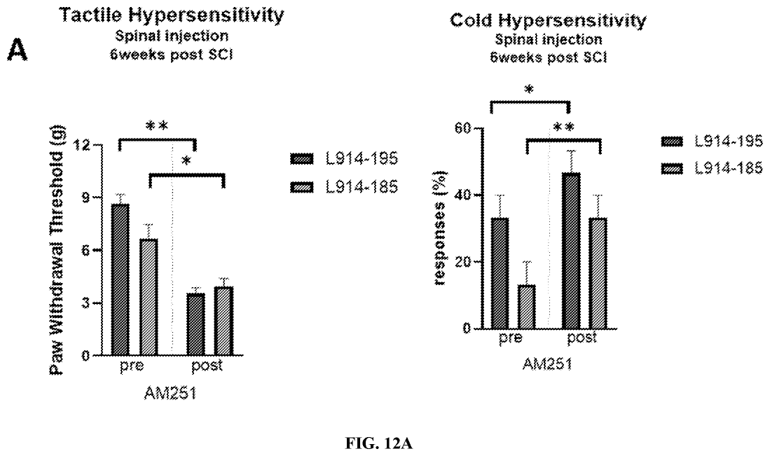

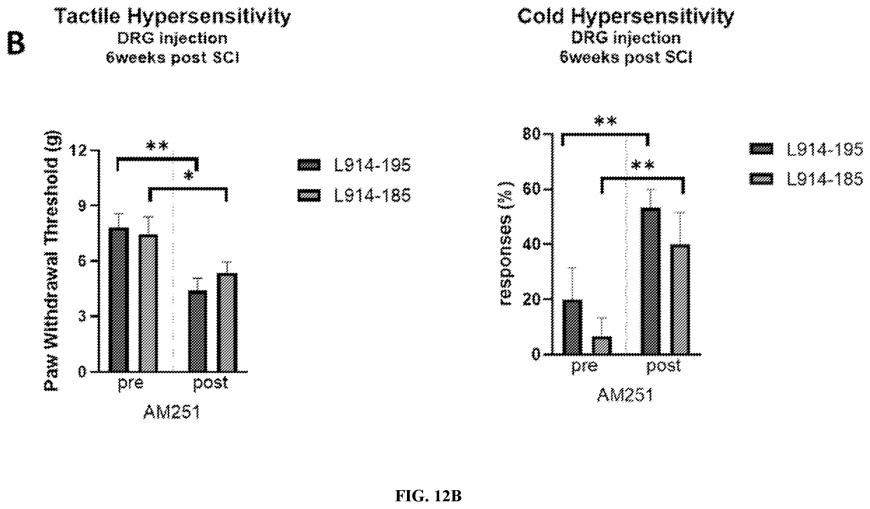

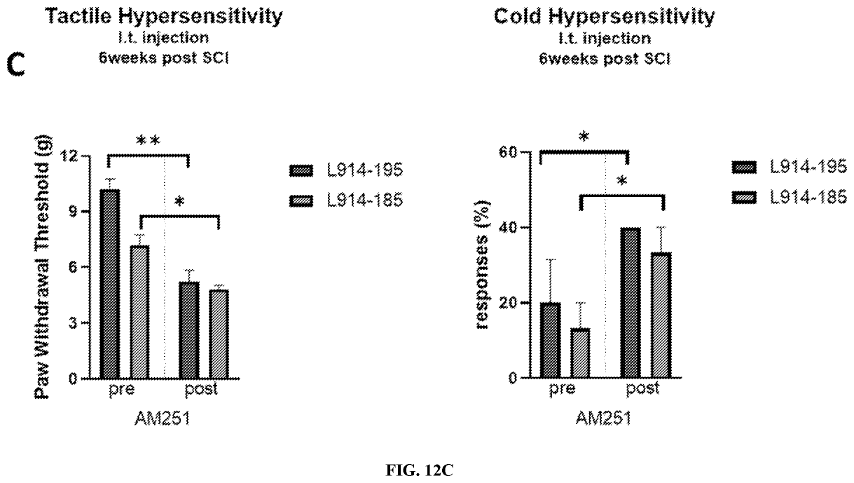

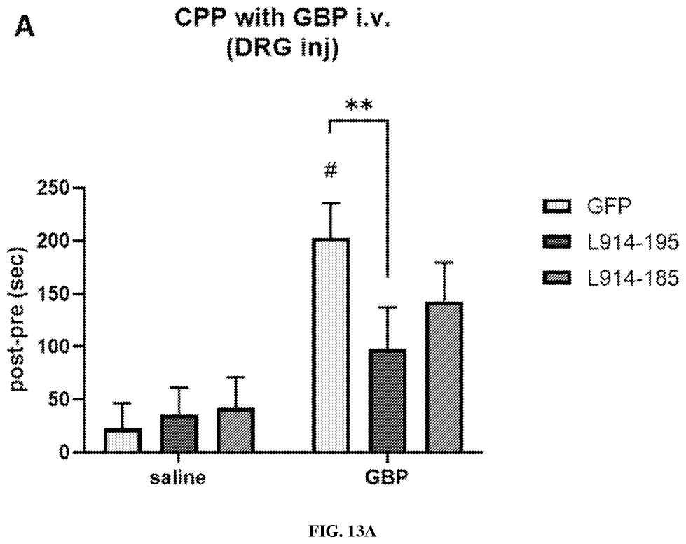

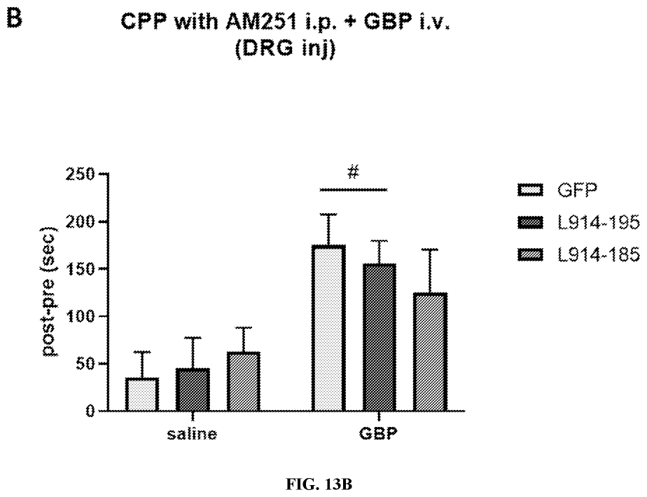

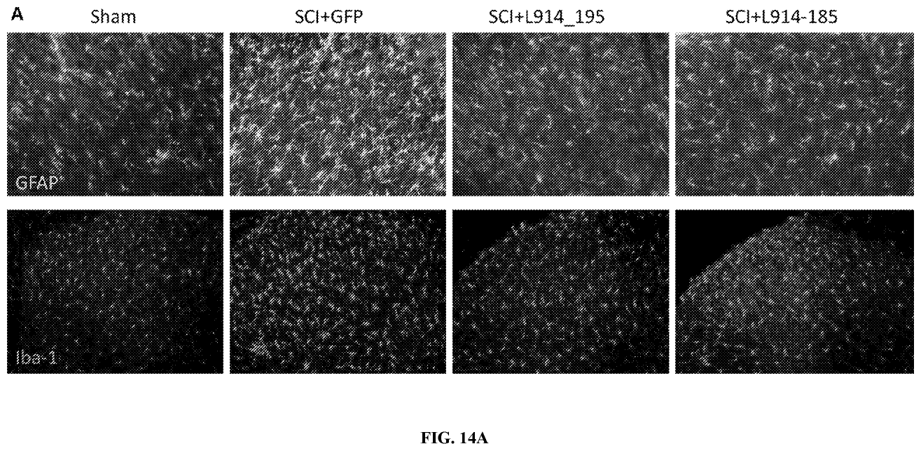

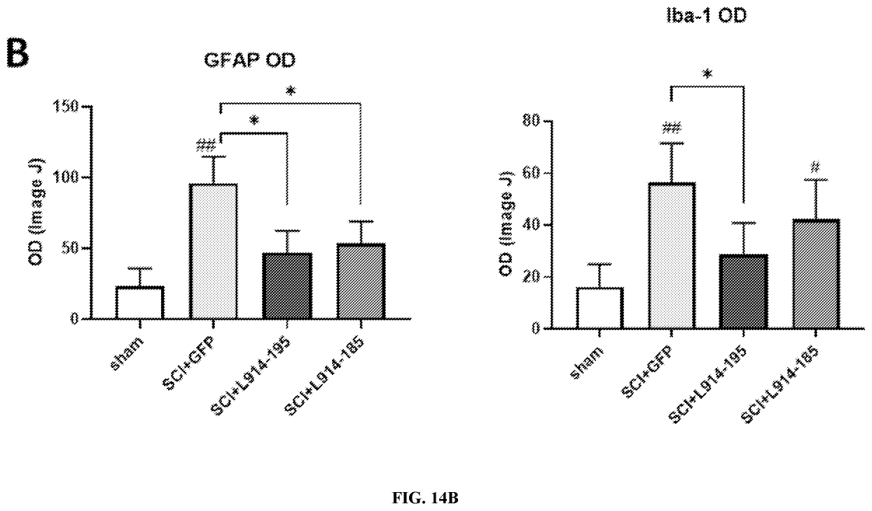

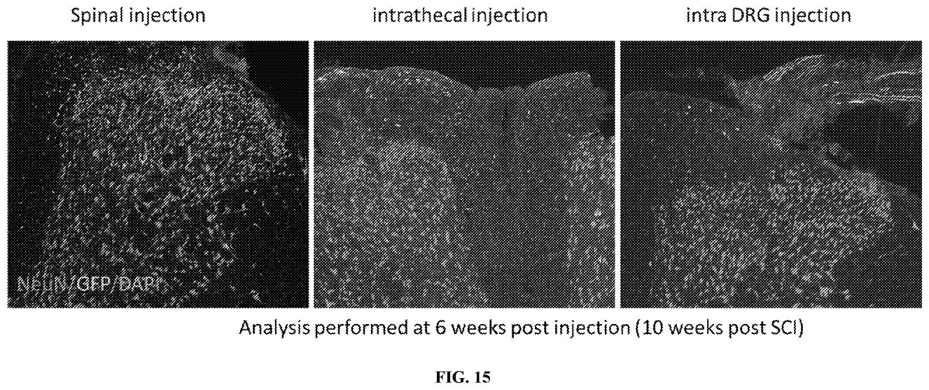

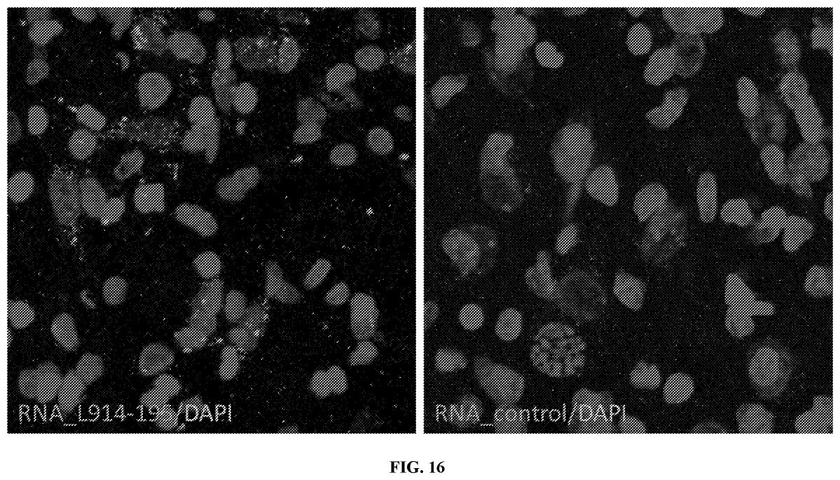

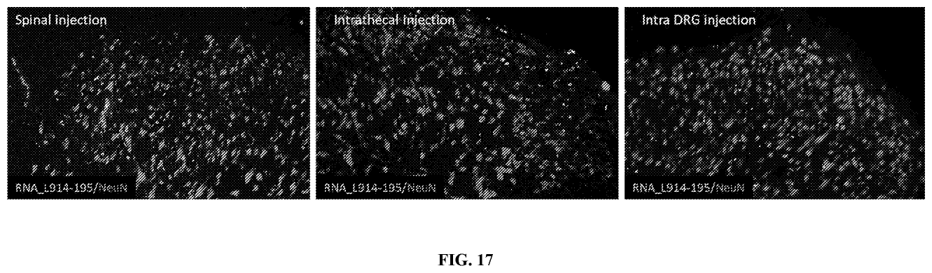

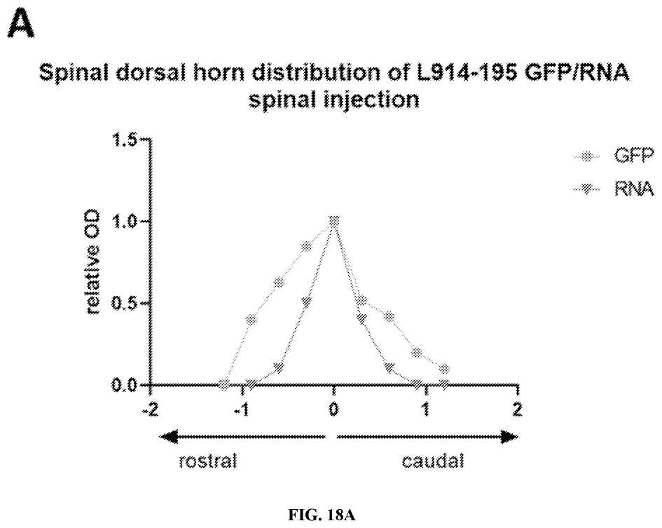

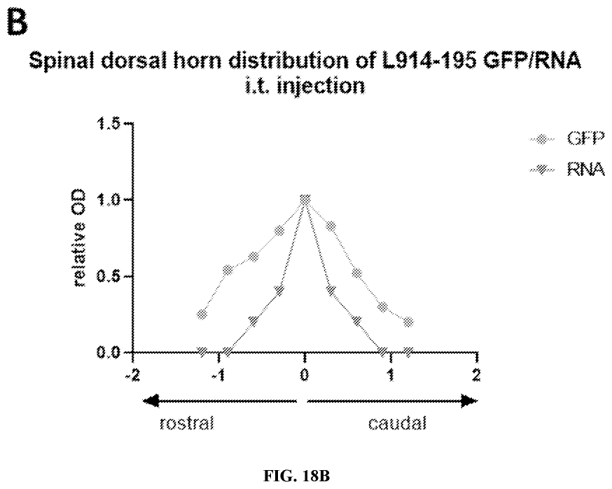

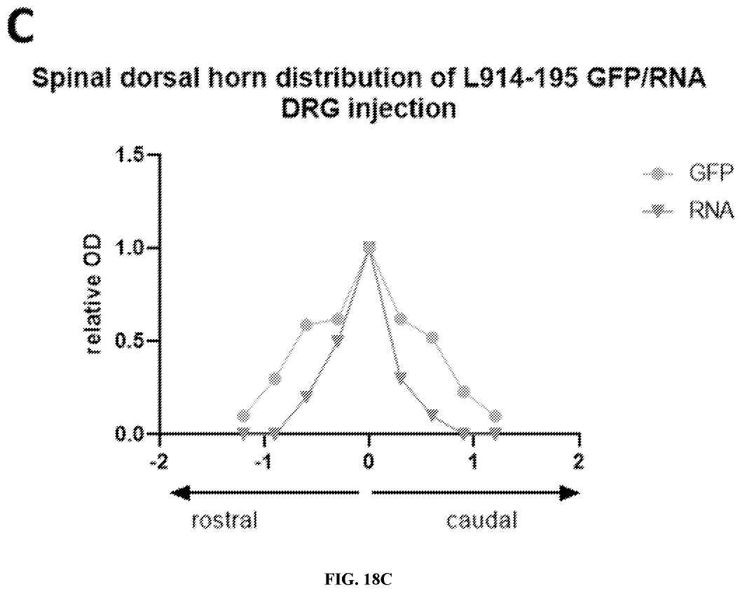

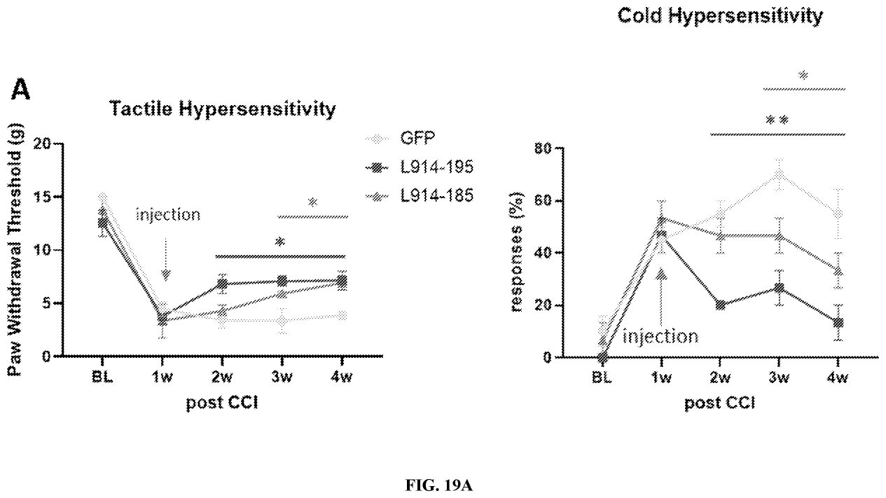

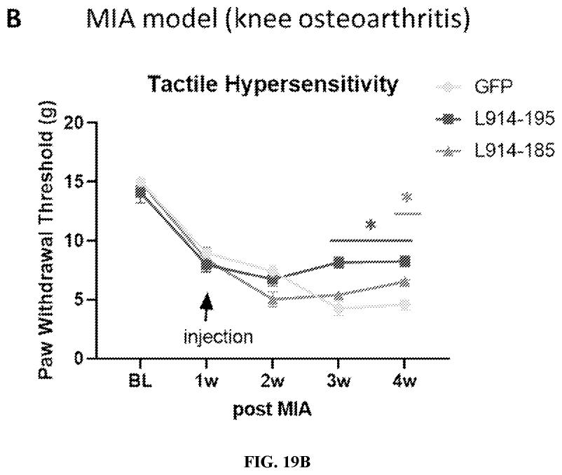

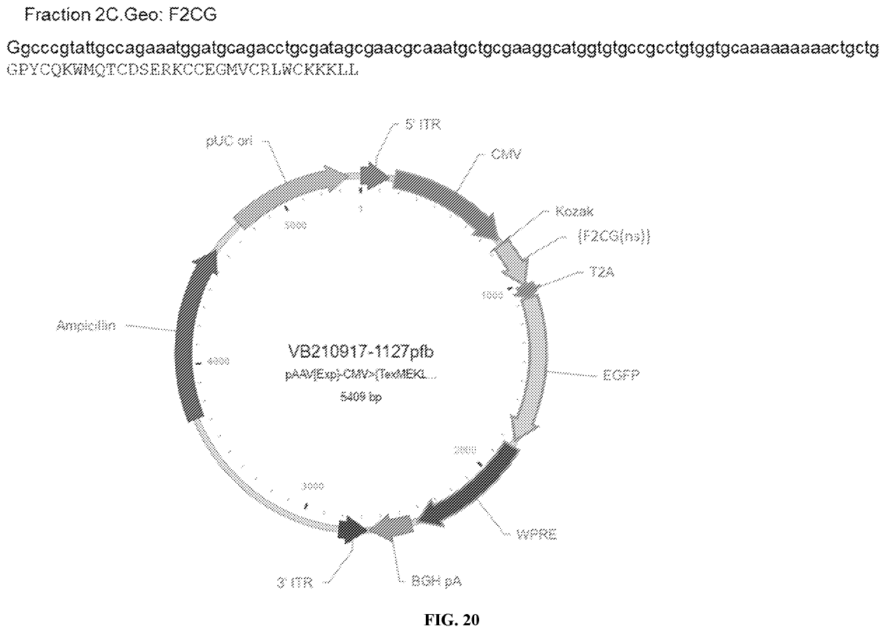

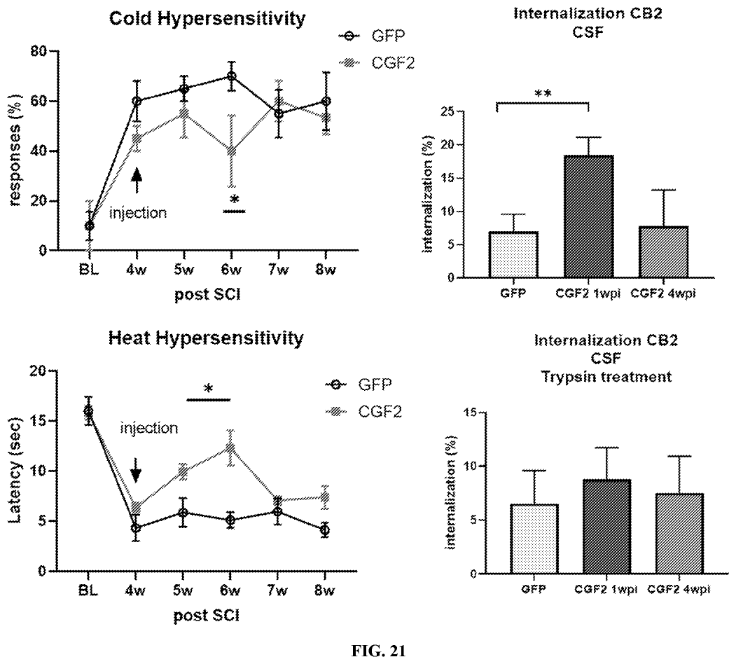

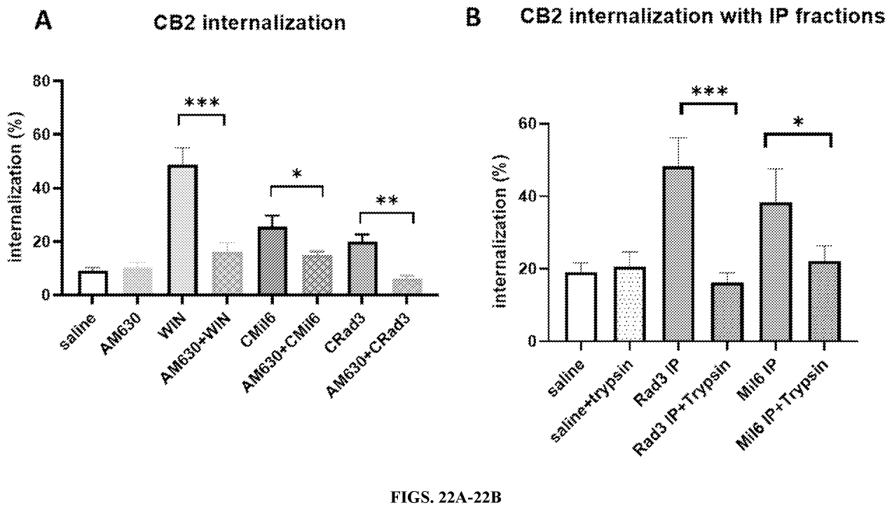

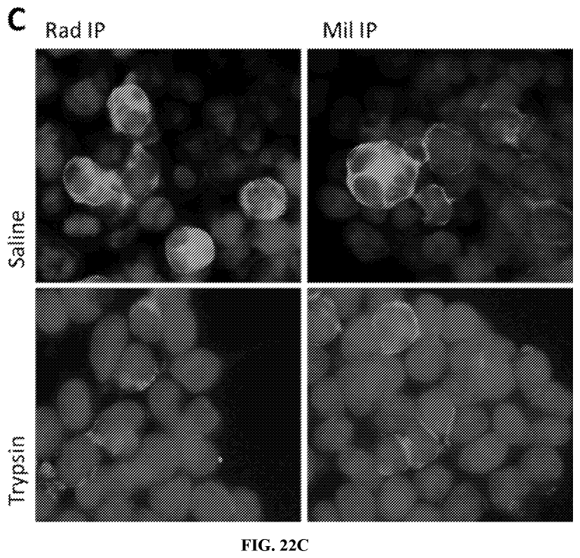

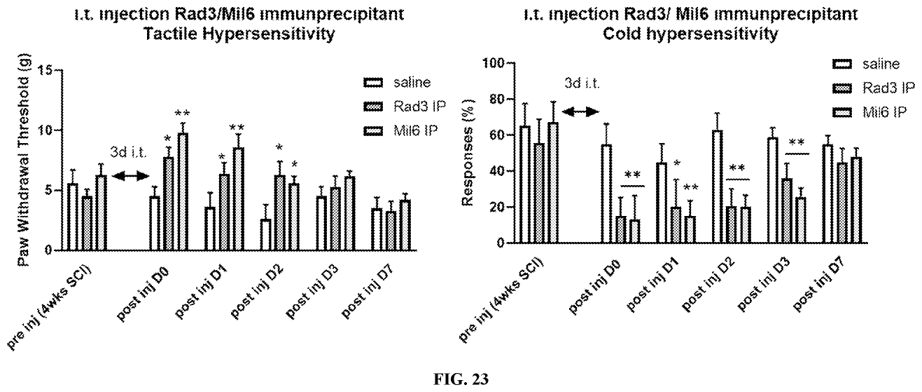

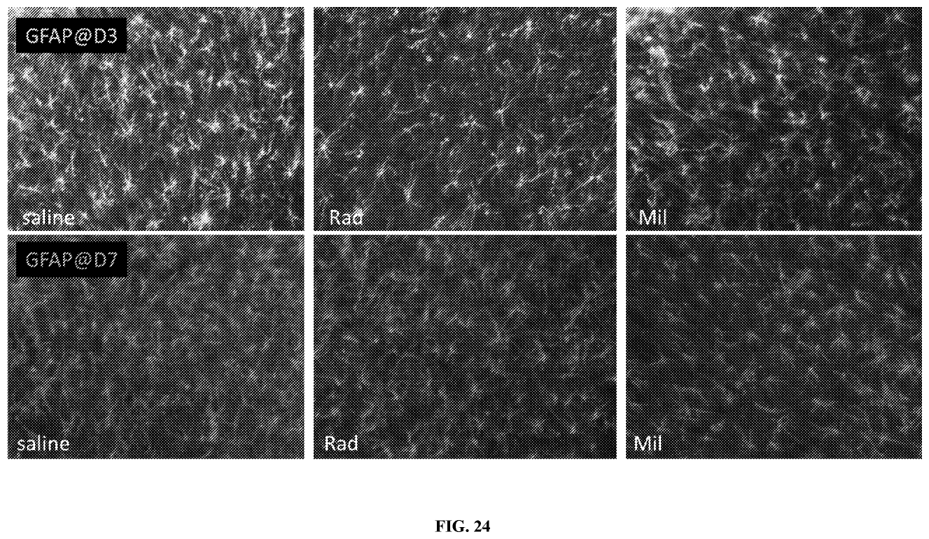

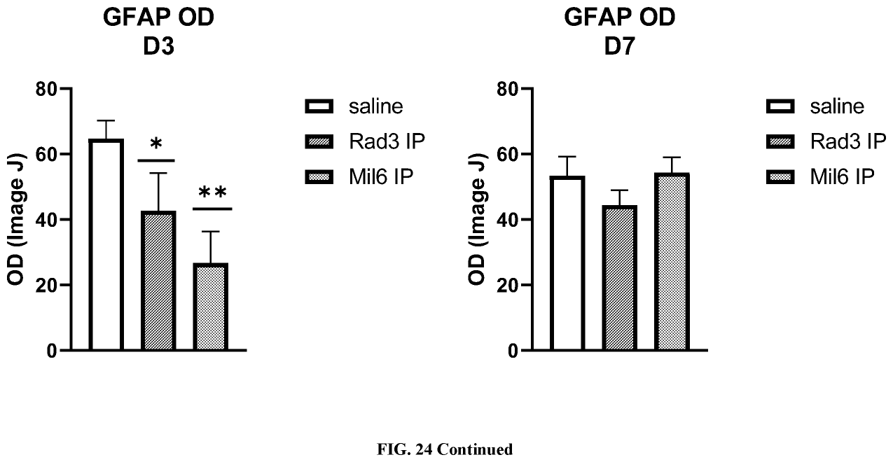

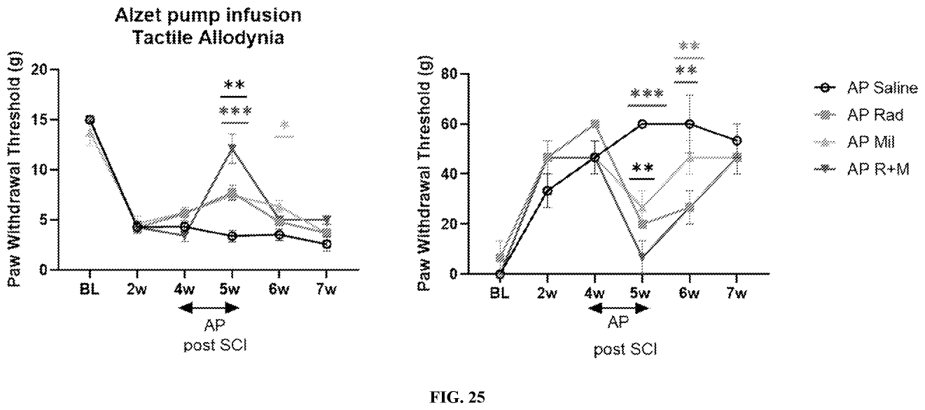

Vector Builder software (Vector Builder Inc.) was used to design plasmids and to generate bacterial colonies with plasmids as well as small samples of viral particles encoding cDNA of selected subfractions. Vectors were designed with mammalian gene expression vector AAV2/8, CMV promoter, T2A linker, EGFP as secondary ORF, and WPRE as regulatory element (FIG. 1 and FIG. 2). The resultant AAV_L914-185 or AAV_L914-195 were injected into the dorsal horn of rats with neuropathic pain following a spinal cord injury (1 μl injected of 10E8 concentration of viral particles in HBSS). Both of these AAVs significantly reduced neuropathic pain in the treated animals, in comparison with control AAVs. At 10 weeks post SCI animals were perfused and their CSF and spinal cord analyzed. Reduced levels of TNFα were detected in the animals treated with L914-195 vector, indicating reduced inflammatory response induced by the analgesic CB1 conopeptide vector. Immunohistochemical analysis of spinal cord lumbar sections showed the presence of eGFP indicating the retained presence of injected vector.

Intraperitoneal injection of CB1 antagonist AM251 (1 mg/kg, Cayman Chemical, MI, USA) led to partial reduction of analgesic affect in L914-195 treated animals when tested for tactile and cold hypersensitivity at 60 min post injection, supporting hypothesized pain reducing mechanisms via CB1 receptor activation by the L914 AAV treatments.

Using similar approaches, a subfraction active on the CB2 receptor isolated from C.Geo fraction 2 (CGF2) was also selected and submitted to Vector Builder to design a plasmid using the same parameters as above. The viral construct was injected intraspinally using the same parameters as above. The effect on behavior was observed within the first 2 weeks post injection with reduction in cold and heat hypersensitivity.

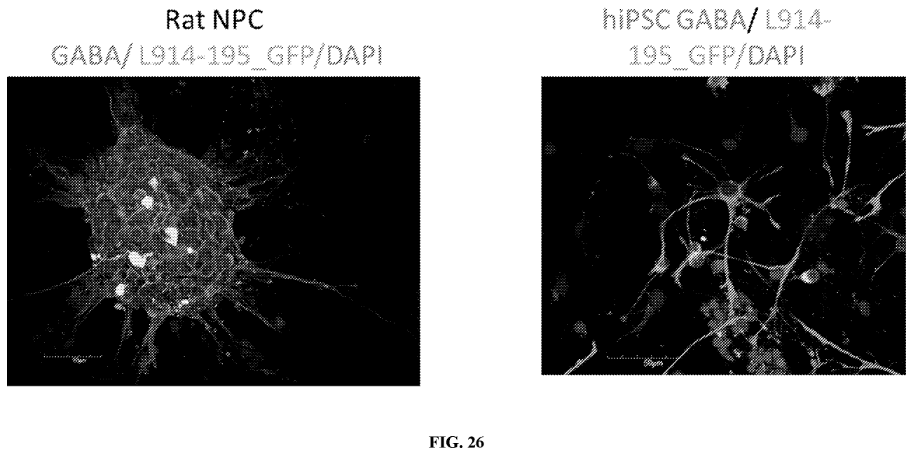

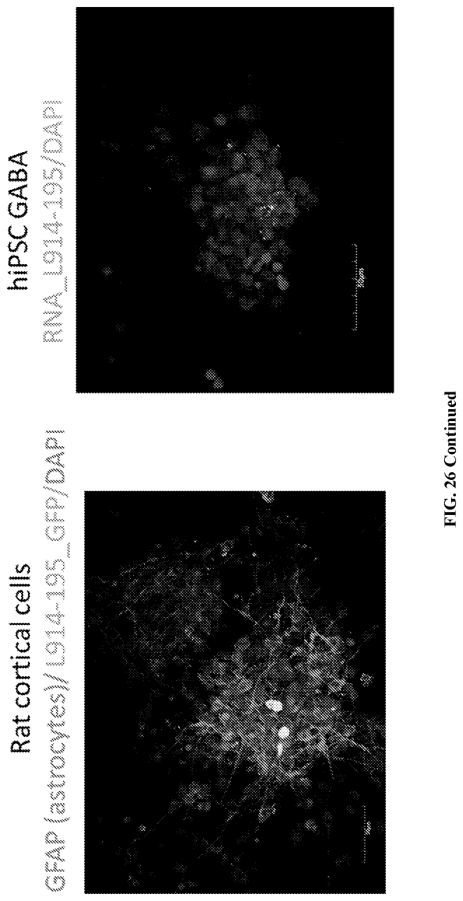

As an alternative approach in achieving sustained production and delivery of the analgesic CB conopeptides, in addition to direct in vivo AAV_CB conopeptide injections, cells may be engineered ex vivo to produce these analgesic peptides followed by transplantation of the transduced cells to the pain-processing regions of the spinal cord or surrounding CSF. To accomplish this, neural stem cells or progenitors were transfected with the AAV_CB1 conopeptide construct L914-195. Both rat neural progenitors (NPCs, E14) and human induced pluripotent stem cells (hiPSCs) have been utilized thus far. Results in rat cultures showed colocalization of the GFP tag with GABA neuronal marker, but no colocalization with astrocytes showing neuronal tropism of the AAV vector. GABAergic neurons derived from hiPSCs were also successfully transduced with the AAV_L914-195_GFP and expressed both GFP marker and L914-195 RNA. These engineered cells can be transplanted for treatment of chronic pain. (FIG. 26.)

### Generating C. Rad3 and C. Mil6 Subfractions

Crude venom extracts from C. miles (C. Mil) and C. radiatus (C.Rad)) were assayed for internalization of CB2 receptor. (The initial venom extracts were a gift from Prof. Baldomero Olivera, University of Utah; Salt Lake City, UT). Fractions were analyzed by CB2 redistribution assay and C. Rad3 and C. Mil6 subfractions were selected as the most promising based on high CB2 internalization, selective CB2 antagonist blocking, and protease degradation indicative of active peptidergic component.

To isolate putative CB2 receptor active components, an immunoprecipitation assay was used with Sepharose G beads coupled to CB2 receptor peptide. The efficiency of coupling was checked on SDS PAGE. Coupled complexes were then incubated overnight with media containing C.Rad3 and C.Mil6 subfractions and the efficiency of immunoprecipitation was analyzed by gel electrophoresis. Bands identified at 40 kDa were purified from the gel using in-gel digestion protocol with bicarbonate/acetonitrile (1:1, vol/vol). After the gel extraction, the supernatant was collected, air-dried and stored in −20° C. until reconstituted for behavioral pain testing and further analysis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES

SEQ ID NO: 1 (Protein, Synthetic)
GCCSDPRCNYAHPAICGGAAGG
SEQ ID NO: 2 (Protein, Synthetic)
GCCSNPVCHLEHSNLCGGAAGG
SEQ ID NO: 3 (Protein, Synthetic)
GPYCQKWMQTCDSERKCCEGMVCRLWCKKKLL
SEQI ID NO: 4 (DNA, Synthetic)
GGCTGCTGCAGCGATCCGCGCTGCAACTATGCGCATCCGGCGATTTGC
GGCGGCGCGGCGGGCGGC
SEQ ID NO: 5 (DNA, Synthetic)
GGCTGCTGCAGCAACCCGGTGTGCCATCTGGAACATAGCAACCTGTGC
GGCGGCGCGGCGGGCGGC
SEQ ID NO: 6 (DNA, synthetic)
GGCCCGTATTGCCAGAAATGGATGCAGACCTGCGATAGCGAACGCAAA
TGCTGCGAAGGCATGGTGTGCCGCCTGTGGTGCAAAAAAAAACTGCTG

1. An engineered conopeptide, wherein the engineered conopeptide is a cannabinoid receptor agonist.

2. The engineered conopeptide of claim 1, wherein the engineered conopeptide is a cannabinoid receptor type (CB) 1 agonist and/or a CB2 agonist.

3. The engineered conopeptide of claim 1, where the engineered conopeptide is derived from a Conus species.

4. The engineered conopeptide of claim 3, wherein the Conus species is C. textile, C. miles, C. quericinus, C. magus, C. geographus, or C. radiatus.

5. The engineered conopeptide of claim 3, wherein the engineered conopeptide is derived from C. textile.

6. The engineered conopeptide of claim 3, wherein the engineered conopeptide is derived from C. geographus.

7. The engineered conopeptide of claim 1, wherein the engineered conopeptide is generated by
a) obtaining a venom sample from the Conus species;
b) separating two or more fractions of conopeptides from the venom sample;
c) performing a fluorescent CB redistribution assay on each of the separated fractions;
d) selecting the fraction with the highest level of fluorescence;
e) isolating a conopeptide from the selected fraction of step d);
f) sequencing the isolated conopeptide; and
g) synthesizing to create the engineered conopeptide.

8. The engineered conopeptide of claim 7, wherein the CB redistribution assay is a CB1 or CB2 redistribution assay.

9. The engineered conopeptide of claim 1, comprising an amino acid sequence at least 80% identical to SEQ ID NO: 1, SEQ ID NO: 2, or SEQ ID NO: 3, or a fragment thereof.

10. The engineered conopeptide of claim 1, comprising the amino acid sequence of SEQ ID NO: 1, SEQ ID NO: 2, or SEQ ID NO: 3.

11. An engineered polynucleotide comprising a nucleic acid sequence encoding the engineered conopeptide of claim 1.

12. The engineered polynucleotide of claim 11, wherein the nucleic acid sequence is at least 80% identical to SEQ ID NO: 4, SEQ ID NO: 5, or SEQ ID NO: 6, or a fragment thereof.

13. The engineered polynucleotide of claim 11, wherein the nucleic acid sequence is SEQ ID NO: 4, SEQ ID NO: 5, or SEQ ID NO: 6.

14. A vector comprising the engineered polynucleotide of claim 11.

15. The vector of claim 14, wherein the vector is a viral vector or a non-viral vector.

16. The vector of claim 15, wherein the viral vector is an adeno-associated virus (AAV) vector, a lentiviral vector, or a herpes simplex virus (HSV) vector.

17. The vector of claim 15, wherein the non-viral vector is a liposome.

18. An engineered cell comprising of the engineered polynucleotide of claim 11.

19. (canceled)

20. (canceled)

21. A method of treating pain in a subject in need, comprising administering to the subject a therapeutically effective amount of the engineered conopeptide of claim 1.

22. (canceled)

23. (canceled)

24. (canceled)

25. (canceled)

26. A method of treating pain in a subject in need, comprising
a) obtaining a venom sample from the Conus species;
b) separating two or more fractions of conopeptides from the venom sample;
c) performing a fluorescent CB redistribution assay on each of the separated fractions;
d) selecting the fraction with the highest level of fluorescence;
e) isolating a conopeptide from the selected fraction of step d);
f) administering to the subject in need a therapeutically effective amount of the conopeptide of step e).

27. (canceled)

28. (canceled)
